(19) United States
(12) Patent Application Publication
(10) Pub. No.: US 2020/0405332 A1
(43) Pub. Date: Dec. 31, 2020

(54) PHARMACEUTICAL COMPOSITIONS AND METHODS FOR THE TREATMENT OF THROMBOSIS AND DELIVERY BY MEDICAL DEVICES

(71) Applicant: Distroller USA, Inc.

(72) Inventor: Michael K. Handley, Fort Collins, CO (US)

(73) Assignee: Marizyme Biotech, Davenport, FL (US)

(21) Appl. No.: 16/457,291

(22) Filed: Jun. 28, 2019

(51) Int. Cl. A61B 17/22; A61K 38/54; A61P 7/02

(52) U.S. Cl. CPC A61B 17/22; A61K 38/54; A61F 2/82; A61B 2017/22084; A61B 2017/22067; A61P 7/02

(57) ABSTRACT

A pharmaceutical composition and a method of using the pharmaceutical composition for the treatment of thrombosis are provided. The pharmaceutical composition can include a mixture of proteolytic enzymes, and optionally, additional compounds. The pharmaceutical composition can include an antiaggregatory or anti-thrombotic compound, such as Lisini racemici acetylsalicylase. The method can include administering the pharmaceutical composition to a patient in need thereof, including administration of the pharmaceutical composition to a thrombus until the thrombus is dissolved. The method can also include administering one or more balloon catheters to the patient.

### CROSS-REFERENCE TO RELATED APPLICATIONS

This application claims the benefit of priority under 35 U.S.C. § 120 of U.S. Provisional Patent Application Ser. No. 62/691,319, filed on Jun. 28, 2018, the content of which is relied upon and incorporated herein by reference in its entirety.

### TECHNICAL FIELD

The present disclosure relates generally to compositions, devices, and methods for the treatment of thrombosis.

### BACKGROUND

Arteriosclerosis occurs when blood vessels carrying oxygen and nutrients from the heart to the rest of the body (arteries) become thick and stiff, which sometimes restricts blood flow to organs and tissues. Healthy arteries are flexible and elastic; but over time, the walls the arteries can harden—a condition commonly called hardening of the arteries. Atherosclerosis is a type of arteriosclerosis that specifically refers to the buildup of fats, cholesterol, and/or other substances in the artery walls (plaque), which can restrict blood flow. The plaque can burst, triggering a blood clot. A blood clot formed in situ within the vascular system of the body and impeding blood flow is called a thrombus. Thus, atherosclerosis can affect arteries anywhere in the body. Atherosclerosis may be preventable and/or treatable, but remains a major cause of death.

Trigger of thrombus in the artery and thrombotic occlusion is a rupture (exulceration) of an atherosclerotic plaque. The sooner the blood flow can be reinstated, the better are the chances are for avoiding damage to heart or brain tissues. Current treatments include mechanical re-canalization (PTCA/PTA+stenting), and thrombolysis (breakdown of the blood clots formed in the blood vessels using medication). The efficiency of re-canalization with current thrombolytics is only about 40-50%. PTA (Percutaneous Transluminal Angioplasty) relates to the mechanical (e.g., catheterization, ballooning) breakdown of thrombus and/or atheroplaque in all vessels. PTCA (Percutaneous Coronary Transluminal Angioplasty) relates to the mechanical breakdown of thrombus and/or atheroplaque in a coronary artery. PCI (Percutaneous Coronary Interventions) relates to an acute procedure to break down coronary thrombus in AIM ( ) or the critical narrowing via PTCA, and is associated with stenting. These techniques are well-regarded for invasive cardiology/angiology, but there are disadvantages. A patient on dual anti-aggregate therapy increases the risks of bleeding (brain, gastrointestinal), which is a contraindication for routine acute operations (e.g., appendicitis, etc.) and operation of accidents (fractures etc.). PCI does not allow an evaluation of proportions between thrombus and arteriosclerosis. Up to 50% of stenting may be avoided.

Current thrombolytic agents include serine proteases that convert plasminogen to the natural fibrinolytic agent plasmin that breaks down the fibrinogen and fibrin contained in a clot. These fibrinolytics can be divided into two categories: fibrin-specific agents, and non-fibrin-specific agents, some of which can catalyze systemic fibrinolysis. Thrombolytic agents can be administered systematically or directly into the thrombus area (Selective Intracoronary Thrombolysis—SIT).

Some current thrombolytic agents are associated with enhanced activity of circulating plasminogen. Risk associated with current thrombolytics is bleeding. The most significant bleeding complication is hemorrhagic stroke, associated with high mortality and long-term disability. Current thrombolytics can also be slow to achieve thrombolysis and re-canalization (e.g., about 30 min). Because time elapse is important to the treatment, (e.g., neurons are harmed after only about 3 minutes; myocardium initial damage occurs within 8 minutes), the use of thrombolytics or thrombolysis has diminished in favor of faster mechanical re-canalizations such as PTA and PTCA. Methods of treating thrombus that are fast, safe, and efficient are needed. Particularly, methods that do not cause bleeding or hemorrhagic stroke.

### SUMMARY OF THE INVENTION

In various embodiments, a pharmaceutical composition comprising an enzyme or a mixture of enzymes is provided. In some embodiments, the enzyme is a proteolytic enzyme. In some embodiments, the mixture of enzymes is a mixture of proteolytic enzymes. In some embodiments, the mixture of proteolytic enzymes are Krill enzymes. In some embodiments, the pharmaceutical composition includes an additional agent, such as an antiaggregatory compound. In some embodiments, the antiaggregatory compound is Lisini racemici acetylsalicylase.

In various embodiments, a method of treating a thrombus in a patient is provided. The method can include the administration of a pharmaceutical composition including an enzyme or a mixture of enzymes to the patient. In some embodiments, the enzyme is a proteolytic enzyme. In some embodiments, the mixture of enzymes is a mixture of proteolytic enzymes. In some embodiments, the mixture of proteolytic enzymes are Krill enzymes. In some embodiments, the pharmaceutical composition can also include an additional compound, including an antiaggregatory compound. In some embodiments, the antiaggregatory compound is Lisini racemici acetylsalicylase. In some embodiments, the method of treatment also includes the use of a balloon catheter. In some embodiments, the method of treatment includes the use of two balloon catheters.

Additional features and advantages of the embodiments disclosed herein will be set forth in the detailed description that follows, and in part will be clear to those skilled in the art from that description or recognized by practicing the embodiments described herein, including the detailed description which follows, the claims, as well as the appended drawings.

Both the foregoing general description and the following detailed description present embodiments intended to provide an overview or framework for understanding the nature and character of the embodiments disclosed herein. The accompanying drawings are included to provide further understanding and are incorporated into and constitute a part of this specification. The drawings illustrate various embodiments of the disclosure, and together with the description explain the principles and operations thereof.

### BRIEF DESCRIPTION OF THE DRAWINGS

A complete understanding of the present embodiments and the advantages and features thereof will be more readily understood by reference to the following detailed description when considered in conjunction with the accompanying drawings wherein:

FIG. 1 illustrates a process of introducing a catheter close to the thrombus using standard procedures like X-ray catheterization.

FIG. 2 illustrates a process of introducing a catheter close to the thrombus using standard procedures like X-ray catheterization, and the delivery of the enzyme composition in the thrombotic vessel via a balloon to dissolve a thrombus.

FIG. 3A is an image of a “fresh” red thrombi (ca 2 days) isolated from a patient with lethal pulmonary embolism.

FIG. 3B is an image of the red thrombi of FIG. 3A, after being dissolved by an enzyme composition, in accordance with embodiments described herein.

FIG. 4A is an image of a several-weeks old thrombus including a substantial amount of connective tissue.

FIG. 4B is an image of the several-weeks old thrombus of FIG. 4A after treatment with an enzyme composition in accordance with embodiments described herein, showing a selective decomposition pattern, with dissolution of fibrin while the connective tissue remained unchanged.

FIG. 5A is a Doppler image of a vessel with a normal blood flow.

FIG. 5B is a Doppler image of a vessel with thrombus with residual blood flow.

FIG. 5C is a Doppler image of a vessel with thrombus after treatment with an enzyme composition, showing the dissolution of the thrombus, in accordance with embodiments described herein.

FIG. 6 shows a histology of an open vessel with the formation of new thrombus 15 min. after treatment with an enzyme composition, and confirming that the enzyme composition does not alter the normal blood forming cascade, in accordance with embodiments described herein.

FIG. 7 illustrates a normal blood flow in a vessel immediately after stent implantation.

FIG. 8 shows the vessel of FIG. 7 ten minutes after stent implantation, with thrombus occluded the stent lumen.

FIG. 9 shows the vessel of FIG. 8, two minutes after the enzyme composition application, with the blood flow in the stent lumen fully normalized.

It will be recognized that some or all of the figures are schematic representations for purposes of illustration. The figures are provided for the purpose of illustrating one or more embodiments with the explicit understanding that they will not be used to limit the scope or the meaning of the claims.

### DETAILED DESCRIPTION

Unless defined otherwise, all technical and scientific terms used herein have the same meanings as commonly understood by one of ordinary skill in the art to which this invention belongs. Although any methods and materials similar or equivalent to those described herein can be used in the practice or testing of the present disclosure, the preferred methods, devices, and materials are now described. All technical and patent publications cited herein are incorporated herein by reference in their entirety. Nothing herein is to be construed as an admission that the disclosure is not entitled to antedate such disclosure by virtue of prior invention.

As used in the specification and claims, the singular forms “a”, “an” and “the” include plural references unless the context clearly dictates otherwise.

As used herein, the term “comprising” is intended to mean that the compositions and methods include the recited elements, but do not exclude others. “Consisting essentially of” when used to define compositions and methods, shall mean excluding other elements of any essential significance to the combination. For example, a composition consisting essentially of the elements as defined herein would not exclude other elements that do not materially affect the basic and novel characteristic(s) of the disclosure. “Consisting of” shall mean excluding more than trace amount of other ingredients and substantial method steps recited. Embodiments defined by each of these transition terms are within the scope of this disclosure.

The term “treatment” or “treating” means any treatment of a disease or disorder in a subject, such as a mammal, including: inhibiting the disease or disorder, that is, arresting or suppressing the development of clinical symptoms; and/or relieving the disease or disorder that is, causing the regression of clinical symptoms.

As used herein, the term “preventing” refers to the prophylactic treatment of a patient in need thereof. The prophylactic treatment can be accomplished by providing an appropriate dose of a therapeutic agent to a subject at risk of suffering from an ailment, thereby substantially averting onset of the ailment. Preventing includes protecting against the disease or disorder, e.g., causing the clinical symptoms not to develop.

It will be understood by those skilled in the art that in human medicine, it is not always possible to distinguish between “preventing” and “suppressing” since the ultimate inductive event or events may be unknown, latent, or the patient is not ascertained until well after the occurrence of the event or events. Therefore, as used herein the term “prophylaxis” is intended as an element of “treatment” to encompass both “preventing” and “suppressing” as defined herein. The term “protection,” as used herein, is meant to include “prophylaxis.”

The term “therapeutically effective amount” refers to an amount of proteolytic enzyme mixture, typically delivered as a pharmaceutical composition, that is sufficient to effect treatment, as defined herein, when administered to a subject in need of such treatment. The therapeutically effective amount will vary depending upon the subject and disease condition being treated, the weight and age of the subject, the severity of the disease condition, the particular compound chosen, the dosing regimen to be followed, timing of administration, the manner of administration and the like, all of which can be determined readily by one of ordinary skill in the art.

As used herein, the term “thrombosis” refers to the formation of a blood clot inside a blood vessel, obstructing the flow of blood through the circulatory system. In some aspects, the thrombosis is “venous thrombosis” which is a blood clot that forms within a vein.

The present disclosure relates to a proteolytic enzyme composition useful for the treatment of thrombus. In some embodiments, the proteolytic enzyme composition comprises a freeze-dried aqueous extract from krill enzymes (e.g., Antarctic, and/or Artic).

In some embodiments, the composition comprises a mixture of naturally-occurring proteolytic enzymes and, optionally, other enzymes. In some embodiments, the composition comprises a mixture of proteolytic enzymes and an antiaggregatory compound, such as, for example, Lisini racemici acetylsalicylase.

In some embodiments, the composition comprises a freeze-dried aqueous extract from krill containing a balanced mixture of naturally occurring proteolytic enzymes, acting in a synergetic manner. The proteolytic enzyme mixture comprises a co-operative multi-enzyme system involving both endo- (trypsin- and chymotrypsin-like enzymes) and exopeptidases (carboxypeptidase A and B). The proteolytic enzymes of the composition mixture may comprise, inter alia, three serine proteinases with trypsin-like activity (two endo/exopeptidases, one endopeptidase); one serine proteinase with chymotrypsin-like activity, four exopeptidases (two carboxypeptidases A and two carboxypeptidases B). The enzyme mixture is mutually protected acting synergistically in a two-step fashion: endopeptidases first attack peptide bonds of the intrastructural parts of the polypeptide chains, and the resulting peptide fragments are subsequently cleaved by exopeptidases into small peptides and free amino acids.

In some embodiments, the proteolytic enzyme mixture is useful for the treatment of thrombus. In some embodiments, the proteolytic enzyme mixture is useful for the treatment of thrombus in vitro, in vivo, and/or in situ, by applying the enzyme composition solution via a device of choice, into the thrombotic vessel until the thrombus is dissolved.

In some embodiments, the proteolytic enzyme mixture may be administered concurrently or subsequently with an antiaggregatory or anti-thrombotic compound. In some embodiments, the antiaggregatory/antithrombotic compound is provided in one vial mixed together with the (natural) proteolytic enzymes. Possible compounds for such use include Lysini Racemici Acetylsalicylas (LRS) (available as Kardegic), Eptifibbatium (available as Intergrilin) or Abciximabum (available as Reopro). Various other antiaggregatory or anti-thrombotic compounds are possible, including:
Cyclooxygenase inhibitors, e.g., Acetylic salicylic acid (Aspirin); Triflusal (Disgren);
Adenosine diphosphate (ADP) receptor inhibitors, e.g., Clopidogrel (Plavix); Prasugrel (Effient); Ticagrelor (Brilinta; Ticlopidine (Ticlid);
Phosphodiesterase inhibitors, e.g., Cilostazol (Pletal);
Protease-activated receptor-1 (PAR-1) antagonists, e.g., Vorapaxar (Zontivity);
Glycoprotein BB/IIIA inhibitors (intravenous use only), e.g., Abciximab (ReoPro); Eptifibatide (Integrilin); Tirofiban (Aggrastat);
Adenosine reuptake inhibitors, e.g., Dipyridamole (Persantine);
Thromboxane inhibitors, e.g., Thromboxane synthase inhibitors; Thromboxane receptor antagonists; Terutroban;
Heparin; and
Tissue plasminogen activator t-PA, e.g., alteplase (Activase); reteplase (Retavase); tenecteplase (TNKase); anistreplase (Eminase); streptokinase (Kabikinase, Streptase); urokinase (Abbokinase).

In some embodiments, Lysini Racemici Acetylsalicylas (LRS), a derivative of acidum acetylosalicylicum (ASA) for intravenous application, is co-administered with the proteolytic enzymes. It is a very efficient antiaggregans with immediate effect after injection. The mode of action is identical to ASA. The indications of LRS are, e.g., acute myocardial infarction, STEMI, unstable angina pectoris, ictus, TIA, etc.

In some embodiments, the pharmaceutical composition comprises about 60 iU of protolytic enzyme mixture and about 900 mg of LRS. The composition may be used to initiate thrombolysis. If needed, additional thrombolysis may be performed using the protolytic enzyme mixture only.

In addition to the active agents, in some embodiments, the composition also comprises fillers, binders, compression agents, lubricants, disintegras, colorants, water, and other elements recognized by one of ordinary skill in the art.

In various embodiments, a method of treating any of the indications mentioned hereinbefore comprising administering to a patient in need thereof a pharmaceutical composition according to the embodiments herein.

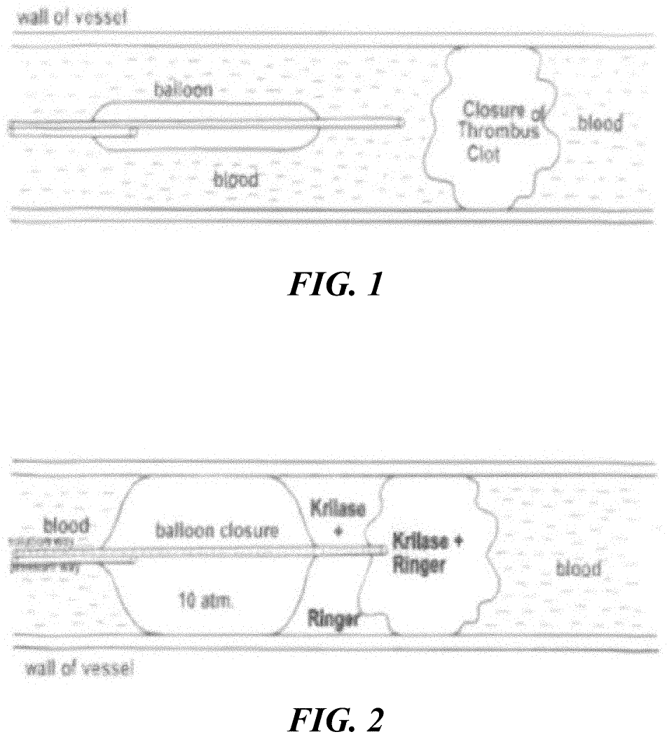

In some embodiments, the fibrinolytic activity of the proteolytic enzymes mixture is ascertained by infusion close or within the thrombus after the blood is removed (washout). This is feasible using a specially designed catheter for the enzyme mixture thrombolysis, as shown in FIG. 1 and FIG. 2. After thrombolysis, the residual atheromatous narrowing may be eliminated via PTCA (near 50% patients). In addition, stenting may be also performed. The proteolytic enzyme mixture can be applied also during vessel dilation (destruction of thrombus and sclerotic plaque), as the enzyme mixture decomposes thrombus detritus as well as detritus sclerotic plaque. The proteolytic enzymes mixture does not affect systemic hemocoagulation. Thus, after local application, the hemocoagulation is also immediately normalized so that a new thrombus formation might take place. Treatment with the proteolytic enzymes does not alter the basic local conditions—it is an ulceration of plaque (coagulation area). To avoid re-thrombotization in the arteries established antiaggregans/antithrombotics should be used preventively. Thus, it is desirable to use a pharmaceutical composition comprising proteolytic enzymes combined with Lisini racemici acetylsalicylase as an optimal drug to prevent re-thrombosis.

### EXAMPLES

The following examples are supposed to further illustrate some embodiments of the disclosure. They are not meant to limit the scope of the claims in any way. One of ordinary skill in the art will appreciate that further developments can be made without deviation from the general idea of the invention described herein.

Safety of Proteolytic Enzymes

The proteolytic enzymes mixture of embodiments described herein demonstrated a broad safety potential with no systemic effects. Thus, there is no risk that the mixture may influence healthy tissues as protease inhibitors in body fluids inactivate them.

Some of the key clinical characteristics of the proteolytic composition mixture include its novel composition, the only product based on a co-operative multienzyme system involving both endo and exopeptidases. The composition has an exceptional safety profile, i.e., when it reaches healthy tissue, the enzymes are immediately inactivated by protease inhibitors; and, the composition has only a limited activity in time and it is rapidly decomposed to harmless basic components like water and soluble amino acids.

The findings above are further strengthen by the fact that high doses of the proteolytic enzyme mixture injected i.v., i.a. or i.m., do not affect the basic physiology or interfere with coagulation blood cascade. Furthermore, experimental and clinical studies show that the proteolytic enzyme mixture is effective and well tolerated without risks for systemic effects.

Experiments were performed on seven pigs (60-80 kg) to ascertain if the proteolytic enzyme mixture may influence basic mammalian metabolism. The animals were continuously monitored. (before the injection and 6 hrs. later). The following parameters were followed: blood pressure, ECG, heart & breath rates. A high dose of the proteolytic composition mixture has been applied (600 U), intravenously or intraarterially. No aberrations from the normal were recorded, leading to the conclusion that the proteolytic enzyme mixture does not affect basic physiology in mammals.

In addition, experiments to establish whether or not proteolytic enzyme mixture affects or influences blood coagulation were also performed. For this purpose, five healthy volunteers (32-75 yrs.) donated 20 ml blood. The samples were separated in two test tubes (10 ml each). A solution comprising 60 U/ml of proteolytic enzyme mixture was added to the first tube, while the second one was used as control. All tubes were stored at room temperature. After 10 minutes, the contents were poured in tray for inspection. In all samples a typical blood coagulum was formed, with similar configuration and strength. The proteolytic enzyme mixture does not affect normal blood coagulation cascade, and it is promptly inactivated by fresh blood.

Thrombolysis in Coronary Arteries, Extremities and Other Arteries Without Angioplasty Using Novel Catheters

The aim of the following studies was to assess the velocity of thrombolysis including, catheterization of proteolytic enzymes in clinical setting. The blood in the vessel was first removed by rinsing with Ringer or physiological solution while the proximal part was blocked by an occlusion balloon. Thereafter, proteolytic enzymes were injected before or directly in the thrombus ascertaining its dissolution. In this setting, the exposure time was not as critical for heart and brain, allowing proteolytic enzymes to act for at least 3 minutes. A typical use of proteolytic enzymes in such a case requires a balloon catheter, to allow both inflation and delivery of the proteolytic enzymes.

The procedure is illustrated in FIGS. 1 and 2, and it is performed in four consecutive steps: (1) Introduce balloon catheter close to the thrombus using standard procedures like X-ray catheterization; (2) Inflate the balloon to achieve closing of the vessel before the thrombus; (3) Directly after closure of the vessel infuse the proteolytic enzymes mixture in a solution into the space between the balloon and thrombus. Infusion press out the remaining blood and consequently a fast thrombolysis is imitated. The infusion may continue until the thrombus is dissolved and the vessel is again fully open; and, (4) Terminate infusion, deflate the balloon and remove the catheter using routine techniques.

In some embodiments, the thrombus may be isolated from both sides. In such embodiments, two balloon catheters may be used to block the vessel upstream and downstream from the thrombus. The two spaces created can be rinsed with ringer solution and then filled with a krill enzyme solution. After thrombolysis the space may be rinsed again before the balloons are deflated in order to allow blood circulation. Advantages of this technique include effectiveness, no remainder of the thrombolysis, and the enzymes will get into the blood stream. This method of treatment is best used in areas that allow to access the thrombus from both sides.

Residual Atheromatous Narrowing After Thrombolysis Be Eliminated Via PTCA (50%), Stenting Could Be Also Performed (20-50%)

If after the thrombolysis, there still remains a significant narrowing of the vessel due to arteriosclerosis (ca 50%) remains, thrombolysis, a regular Percutaneous Transluminal Angioplasty (PTA) or Percutaneous Transluminal Coronary Angioplasty (PTCA) may be also performed.

In some embodiments, a novel balloon catheter was applied in a step-by-step procedure as described in FIGS. 1 and 2. Additionally, the removal of blood close to the thrombus was closely monitored in order to minimize possible inactivation of the enzyme mixture by the blood residues.

Experiments

The chosen animal model (domestic pigs) for thrombolysis because of lower extremities mimics a common human condition. The pigs have weight 70 kg and similar histology of vessels allowing use of established equipment and medication.

The aim was to assess proteolytic enzymes in PTCA/PTA, after the functionality of specific ballooning catheter, and the efficacy of dissolving thrombus and atheroma detritus from the procedures (PTCA+stenting).

The inactivation by blood of the proteolytic enzyme mixture constitutes an advantage for thrombolysis. PTCA and PTA, referred also as coronary artery ballooning and stenting have become one of the common medical interventions performed for coronary artery blockages. By balloon angioplasty atheromatous plaque is compressed and the vessel is stretched resulting in enlargement of the lumen and its outer diameter. The balloon inside the artery is inflated and deflated (up to 20 atm), to compress the blockage against the artery wall and widening the artery so blood flow improves. A stent may be placed within the coronary artery to keep the vessel open. Microembolization of plaque debris and adherent thrombus cause complications by reducing the blood flow resulting new ischemia in the periphery of the tissue.

The fast fibrinolysis provided by the proteolytic enzyme mixture would eliminate the side effects via efficient removal of post-angioplasty residues and consequently by radically improving blood flow and limiting associated tissue ischemia. Using PTCA a time factor is important with a max treatment time of about 3 minutes.

The application of the proteolytic enzymes was similar to the procedures of thrombolysis (see above). The enzymes were injected after a short rinse with solution during balloon inflation and consequent dilatation of coronary artery and stenting. The whole procedure, inflation/deflation 2-3 times required only about 3 minutes.

The aim of this study was to eliminate thrombus and sclerotic plaque residues in ischemia vulnerable localizations like brain or heart using the enzyme mixture. Moreover, also preventive measures of embolization were investigated via PTCA/proteolytic enzyme mixture. Thrombolysis was run for 3 minutes, mimicking a critical time of irreversible damage of brain tissues.

These experiments were also performed on an animal model (domestic pigs). The efficacy of the proteolytic enzyme mixture was measured by established technologies like angiography, sonography with Doppler, photographic documentation, biochemical (before and after) and histological analyses before and after thrombolysis. Further, the blood was filtrated after the enzyme treatment and the amount of residual debris was zero.

Avoiding Re-Thrombolization

To prevent re-thrombosis, anti-thrombotic therapy should be used until complete endothelial healing. It has been found that the proteolytic enzyme mixture does not influence hemocoagulation, proven by earlier in-vitro and now further in vivo (sonography with Doppler and angiography). As explained in the embodiments above, it has been found that in order to avoid re-thrombosis thrombolytic krill enzymes may be combined with anti-aggregatory compounds such as Lisini racemici acetylsalicylase. By using such a combination, the anti-aggregatory action is secured.

Drug elusion (DE) Coating Combining Proteolytic Enzymes and Cytostatic

PTCA and stent implantation damages the vessels (mainly stratum intimae). Lack of endothelial coverage on such a large surface (2-5 cm2) results in a fast thrombus formation. To avoid such a condition often a dual anti-aggregatory treatment (ACP+Clopidogrel) is administered. However, this approach may cause serious side-effects like bleedings etc.

Moreover, a traumatized vessel heals via formation of tendon causing narrowing of the lumen (tendon stenosis). By applying DE-covered balloons containing cystostatic (e.g., Paclitaxel) the fast tendon ingrowth is prohibited.

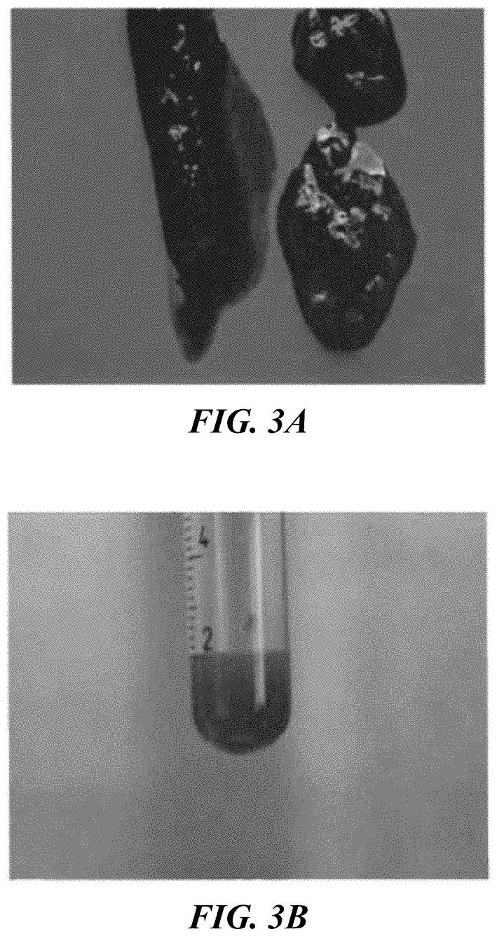

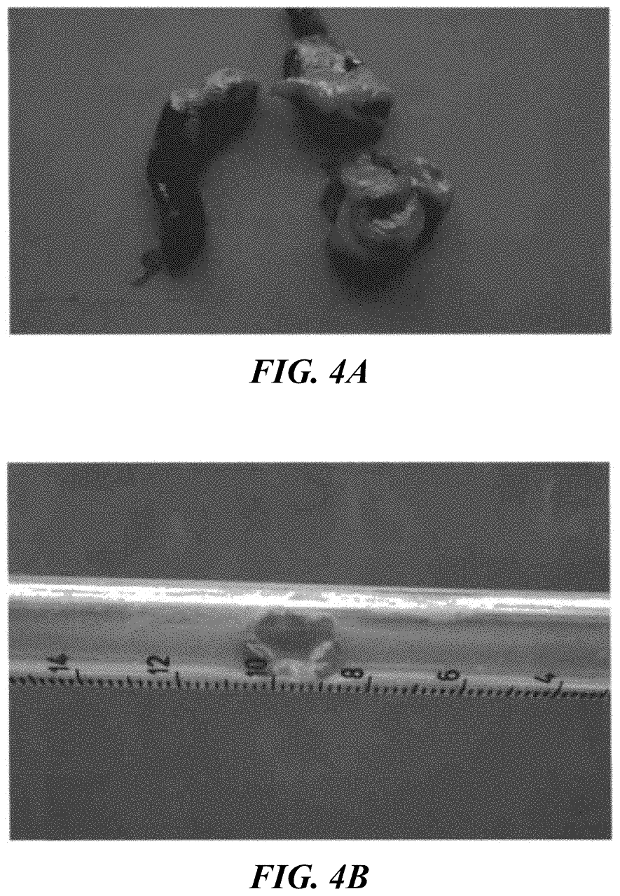

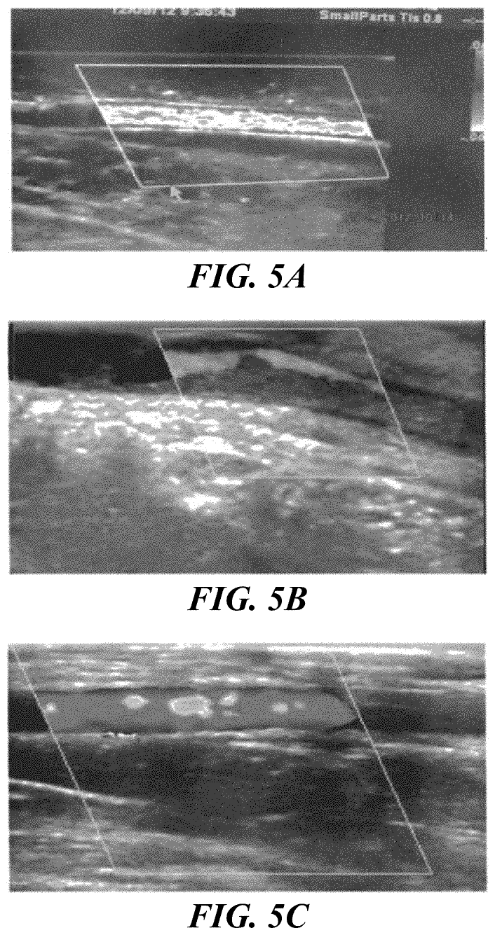

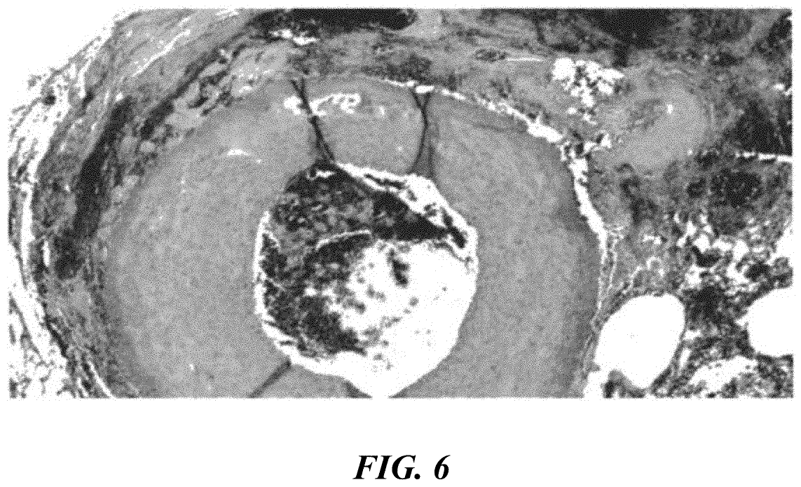

The exceptionally fast proteolytic enzymes mixture thrombolysis was proven both in vitro (FIGS. 3A, 3B, 4A, 4B) and in vivo (FIGS. 4A, 5B, 5C) substantiating that, e.g., a thrombus of 1 cm3 is dissolved in less than 3 min. Thrombus degradation is basically a breakdown of fibrinous matrix that is successively dissolved (supported microscopically, FIG. 6) without residual fragments.

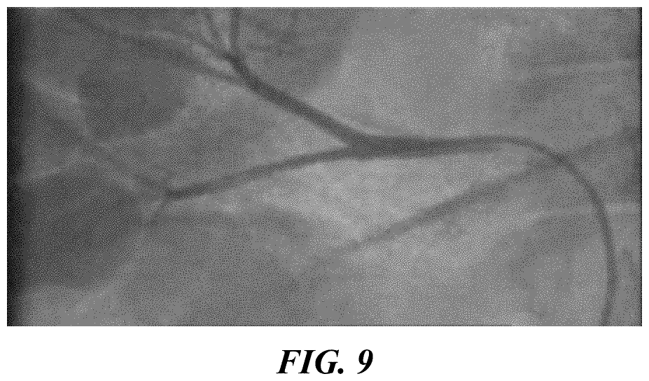

With respect to thrombolysis of “old” thrombi, a deposit of tendon (stroma) will remain attached on vessel's wall (FIGS. 4A, 4B), while the fibrinous matrix of thrombi is dissolved and washed away by blood (FIG. 9). The fast proteolytic enzyme thrombolysis allows an immediate judgment of the stenosis status before a decision of mechanical re-canalization (PTCA, PTA) or stent implantation. In this way the numbers of stenting may be reduced up to 50%.

In some embodiments, to achieve optimal use of the proteolytic enzyme mixture, a novel catheter was designed to avoid the enzyme mixture inactivation by blood.

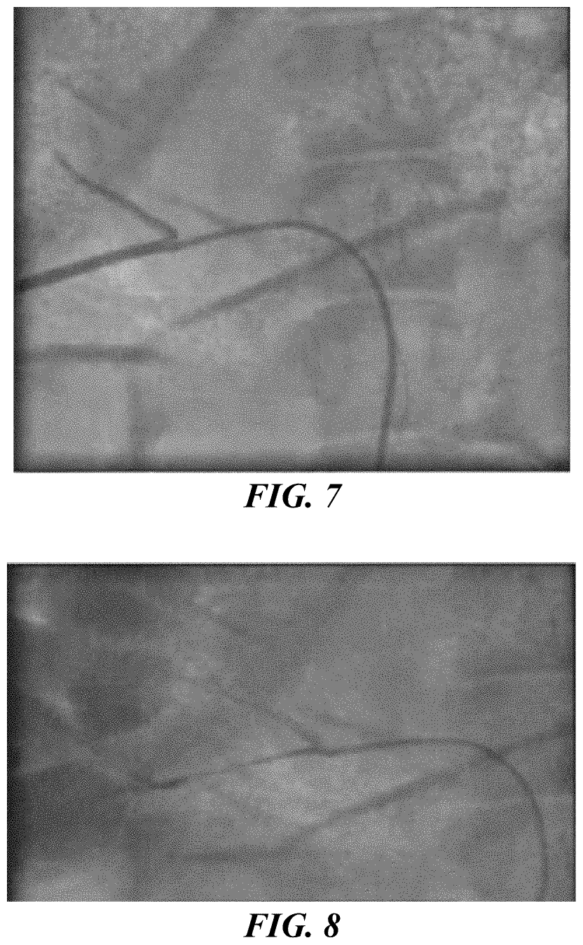

As shown in FIG. 7, the proteolytic enzymes do not affect systemic and local haemocoagulation. Still as shown in FIG. 7, after thrombolysis, an ulceration plaque with coagulation area of 2-5 mm2 remains, contributing to new thrombus formation and re-thrombosis. When combining the proteolytic enzyme mixture with an antiaggregatory compound (e.g., Lisini racemici acetylosalicylici) the risk of rethrombosis is eliminated. The proteolytic enzyme mixture acts as a thrombolyticum, independent of blood factors (plasminogen). This unique characteristic may be exploited by covering biodegradable polymers with cytostatica (like Paclitaxel, Sierolimus, etc.) to the stent (thus forming a Drug Eluting Stent (DES)) to prevent tendon stenosis, as shown in FIG. 9 Catheters or stents combined with these cytostatics are called DE-K (FIGS. 6, 7, 9).

The advantages of the current disclosure include: rapid re-canalization without traumatization vs PTCA or PTA; more gentle—not damaging the vessels; minimize coagulation area vs PTCA and stenting; reduced need of stenting (ca 50%); and no disturbance in hemocoagulation.

Proteolytic enzymes meet the most important requirements for recanalization: rapid onset (ca 3 min, thus 10 times faster than the marketed thrombolytics); selective—not affecting native tissues, only degrading non-viable plaque/thrombus; not interfering with haemocoagulation cascade (in contrast to available thrombolytics) implying low side-effects ratio; no enlarging endothelial surface (compared to PCTA/PTA/stenting).

Until now enzymes could not be used in clinical praxis because there was no way how to prevent its inactivation by blood. The current embodiments offer an innovative solution to overcome this setback.

Earlier the thrombolytic/fibrinolytic potential of proteolytic enzymes has been studied in standard model (Chandler loop assay including human plasma mixed with trace amounts of 125I-labelled human fibrinogen) and was used for evaluation of thrombolytic agents such as streptokinase or tPA (ref). The proteolytic enzyme mixture had the most rapid clot lysis observed. Moreover, the proteolytic enzymes also demonstrated a fast dissolution of thrombi isolated from human cadaver. Two types of thrombi were exemplified: the first one “fresh”, just a few days old “red” thrombus (FIG. 3A) and the second one several weeks old thrombus including substantial amount of connective tissue (FIG. 4A).

Both samples were treated with proteolytic enzymes and the results were in line with the previous in vitro data pointing to fast thrombolysis for the fresh thrombus (dissolved within 3 min, FIG. 3B) while the old thrombus demonstrated a selective decomposition pattern, namely similar dissolution of fibrin whereas the connective tissue remained unchanged (FIG. 4A). The connective tissue is closely associated with the vessels, thus not causing risks of embolization.

Based on the above experiments, the in vitro the activity of the proteolytic enzymes was also studied in vivo (rabbit). It was further shown that proteolytic enzymes were effectively inactivated by plasma inhibitors. These data confirmed the overall safety profile for the proteolytic enzyme mixture in clinical applications.

Two distinct features characterize Krill enzymes, namely highly efficient and rapid effect onset in vitro and complete inhibition in vivo (important safety aspect). Paradoxically, these two seemingly contradictory properties may open an important niche for use proteolytic enzymes in treatment of cardio-angina issues.

Sonography with Doppler

For this study an animal (pig) model was chosen due to its similarities (biochemical, hematological and immunological) with humans. The study was performed on 3 pigs, approx. weight 70 kg, in accordance with EU regulations.

The testing was performed by a team including veterinary surgeons, anesthesiologist, and specialists on modern monitoring methodologies monitoring the blood flow like sonography and Doppler. In each animal a surgery ascertained access to 4 arteries and one vein. The animals were anesthetized according to a standard protocol. Thus, ECG, O2, CO2, breathing frequency, etc. were continuously monitored. After the experiments, euthanasia was performed following EU directives. Thrombus was formed via mechanical damage of the vessel (disintegration of intima). The thrombus formation was accelerated by addition of small amount of thrombin (0.1 cc) resulting in a solid thrombus within ca 20 min. Proteolytic enzymes were injected (0.5 ml) after the blood was rinsing from the vessel. The blood flow, thrombus formations as well as the course of thrombolysis with the flow re-start were monitored by sonography and Doppler. The course of all experiments was documented photographically and followed histological analyses (FIGS. 5A, 5B, 5C and 6). Histology of open vessel (FIG. 6) was performed, visualizing formation of new thrombus 15 min. after treatment with the proteolytic enzyme mixture, confirming that Krill enzymes does not alter normal blood forming cascade. The average time of complete thrombolysis with proteolytic enzymes was 3 min. (range from about 2 min. to about 4 min.). This is a significant improvement over current treatments like, for example, Streptokinase or tPA, which require a duration of at least 30 minutes. After the vessel opening, the rest-products of thrombolysis were washed-out. No solid residues of the thrombus (detritus) were observed. In addition, it was verified that the proteolytic enzymes are inactivated by blood and consequently the thrombolysis ceased. Thereafter, when blood was removed, the thrombolysis could proceed via a new application of proteolytic enzymes, thus confirming that proteolytic enzymes do not alter normal blood forming cascade. This contrasts to current thrombolytics treatments that are causing serious bleeding complications both locally and systemically (brain hemorrhage, contraindication for emergency surgery, etc.).

No clinical side effects were observed (blood pressure, heart rate, allergic reactions, etc.). The laboratory results (biochemistry) were normal (before, during and after the operation).

The resulting data reveals a fast-thrombolytic effect of proteolytic enzymes in vivo compared to current thrombolytics like Streptase or tPA. Moreover, the proteolytic enzymes treatment was safe, not causing bleedings or affecting normal local or systemic coagulation.

A follow-up of previous investigations, using surgery techniques and documentation with sonography and Doppler, a complementary study applied technologies currently used in clinical praxis—namely catheterization angiography. This approach is considered a “gold standard” to assess thrombus formation and vessel re-canalization in human medicine.

The study was performed applying regular clinical equipment and monitored by angiography and the whole procedure was digitalized and saved on DVD(s).

A stent was implanted in the test vessel resulting in endothelial disruption and traumatized surface. Thereafter a balloon was inflated in the stent vicinity so that the lumen was not completely closed but only slowdown the blood circulation. As next step, thrombin was added to enhance solid thrombus formation (within ca 5 min). A complete vessel closure was verified by angiography. A, proteolytic enzymes solution (5 ml) was continuously injected under 1 min. adjacent to the thrombus. The continuous proteolytic enzymes injection in a vessel with only limited blood inflow resulted in complete blood elimination close to the thrombus. The thrombus was dissolved within about 3 min followed by normalized blood circulation. A whole schedule was monitored by angiography. See FIGS. 7, 8, 9.

Additionally, a large supply vessel containing multitude ramification was chosen and a stent was implanted in one of the branches. Thereafter this supply vessel was mechanically blocked by catheter in a wedge position. As above thrombin was added and following 6 min all the vessels network was completely blocked by thrombi and consequent hold up of blood circulation. Proteolytic enzymes (5 ml) were slowly injected in such a large supply vessel and just after about 4 min the whole vessel network was cleared and the normal blood circulation was verified by angiography, saved on DVD.

These examples confirm the proteolytic enzymes mixture unique fibrinolytic and/or thrombolytic activity, as also verified previously in findings in-vitro and in-vivo by means of sonography with Doppler, surgery, and histology (FIGS. 5A, 5B, 5C and 6). Adopting the current techniques (catheterization/angiography) used in clinical praxis, clearly revealed the proteolytic enzymes thrombolytic potential (FIGS. 7, 8, 9).

Novel catheters as outlined in this disclosure should allow optimal use of proteolytic enzymes in clinical praxis. Further, experimental data verified that the proteolytic enzymes do not affect normal haemacoagulation cascade, a combination with antithrombotic drugs would prevent re-thrombosis.

The cumulated experimental data of the proteolytic enzymes mode of action shows that after successful thrombolysis it may be necessary to add antiaggregants to prevent re-thrombosis. Compared to PIC, causing large local damage and fast re-thrombosis, proteolytic enzymes with effective, e.g., Lysini racemici acetylsalicylase, eliminate re-thrombosis.

In some embodiments, the use of any of the selected pharmaceutical compositions comprising the proteolytic enzyme mixtures of the above-referenced embodiments in combination with one or more medical devices.

In some embodiments, methods of delivering a pharmaceutical composition for the treatment of thrombus is provided. In some embodiments, a proteolytic enzyme composition is delivered to human vessels that contain new or aged thrombus in an effort to breakdown the thrombus to provide therapeutic effect of increased profusion.

In some embodiments, a prerequisite for thrombosis therapy may include the targeted thrombus be reachable via the catheterization, such that the balloon catheter is able to block the blood stream in a vessel that is blocked by a thrombus, thus creating a small space (space is only 2-5 cm3) which can be rinsed (e.g., by saline or Ringer solution).

In some embodiments, a balloon catheter blocks the blood stream in a vessel that is blocked by a thrombus, thus creating a small space (space is only 2-5 cm3) which can be rinsed (e.g., by saline or Ringer-solution) and in which the proteolytic enzymes mixture solution can be applied. In some embodiments, the proteolytic enzyme solution contains 6 Units/ml solution. The identified space is rinsed, essentially free of blood components that could inactivate the proteolytic enzymes. If necessary, the thrombus may be rinsed again and the proteolytic enzymes solution may be re-applied. As demonstrated below, a thrombus with a volume of 10 mm3 dissolves within 3 minutes by the application of proteolytic enzymes solution which is much faster than previous reports using different thrombolytics. In addition, there is the advantage that due to the blocked blood flow (by the balloon catheter) there is no risk that any thrombus parts would move from the site and therefore lead to embolization. Further advantages of the treatment are described below.

In some embodiments, a small diameter, multi-lumen catheter may be used. Barium filled polymers, particularly in urethanes that soften at body temperature, are ideal for peripherally inserted lines and drainage catheters. Increased pushability to reach more distal vascular regions for angiographic imaging or therapeutic ablation will benefit from a wide selection of devices now reach smaller vascular pathways in and around the heart to deploy balloons based on polyamide-based polymers with bismuth radiopacifiers.

In some embodiments, the catheters could easily be implemented in existing production lines. The production approaches might vary between the different companies but the outcome is expected to be the same. The catheter including the balloon may be made from currently used materials and approved by health authorities like Duralin®. In some embodiments, the catheter has two functions and therefore includes two tubes, first for inflation of the balloon, and second for rinsing. As shown in FIGS. 1 and 2, the balloon catheter will be inflated by a first tube from the end which is distant from the thrombus and that the outlet of the second tube is located between the thrombus and the balloon. In some embodiments, the balloon is elongated. For example, the size of catheter should correspond to standard use catheters; e.g., a length of about 100-120 cm, a thickness of about 5-7 French. Low pressure occlusion/closure balloon, e.g., length 1 cm, cross-section diameter 3 or 20 mm, after inflation. The catheter final design must be adopted to the indication/localization (coronary artery, carotis art., art. femoris etc.). Further the catheter may be manufactured in different thicknesses adopted to indications (coronary or femorary vessels, brain artheris, etc.).

In some embodiments, the proteolytic enzyme composition mixtures may be delivered in situ using ultrasound to treat endovascular thrombus. In some embodiments, the proteolytic enzyme composition mixtures may be delivered in situ, by using a pulsing laser to provide a photoacoustical effect and treat endovascular thrombus.

In some embodiments, the proteolytic enzyme composition mixtures are delivered using cavitation, directly to the endovascular thrombus.

Furthermore, an energy source (e.g.), if directed at the thrombus, may break the thrombus apart and provide additional surface area for the proteolytic enzymes to work on.

In addition, various devices may be used to deliver the proteolytic enzyme(s), but such devices should contain a biocompatible catheter with a cavity or specifically radial lumen that is large enough to deliver a solution containing the protolytic enzyme mixture. The catheters may also be capable of delivering acoustical energy or laser energy. Furthermore, the catheter may have a semipermeable membrane at the end of the catheter that can allow for the release of the enzyme(s) provided it has a molecular weight cut-off larger than the molecular weight of the enzyme(s). This membrane may also be elastic, so it may be enlarged by inflating with solution of enzyme(s) to occlude the vessel.

In some embodiments, for the stabilization and/or penetration of the enzyme(s) the preparation of the enzyme material may be encapsulated with a rapid dissolving high molecular weight polymer prior to injection. In some embodiments, for the stabilization and/or penetration of the enzyme(s), the preparation of the enzyme material may be co-precipitated with a carbohydrate such as starch prior to injection. In some embodiments, for the stabilization and/or penetration of the enzyme(s), the preparation of the enzyme material may be made into lipid-containing micelles prior to injection.

In various embodiments, a process of extracting a natural proteolytic enzyme mixture form raw krill material is provided. The raw krill material, originating from commercial catches, is frozen immediately and maintained at −20° C. until used. Before use, the blocks are thawed and homogenized in distilled water. Such an aqueous crude extract is defatted and further purified by gel filtration. Fractions containing substances with molecular weights of 20-40 kD are pooled and concentrated by ultra-filtration. The purified extract is subjected to an aseptic manufacturing process including membrane filtration, filling in glass vials and freeze-drying. Usually the product is used in 60 Units per vial (buffered with Trometamol to pH 7.5) which is reconstituted with 10 ml of 0.9% aqueous sodium chloride solution. The product is well characterized with respect to proteolytic activities, batch-to-batch variations and uniformity. The stability of the freeze-dried aqueous extract is excellent. When stored in a cool place (3-8° C.) the shelf life is at least two years.

It will be apparent to those skilled in the art that various modifications and variations can be made to embodiments of the present disclosure without departing from the spirit and scope of the disclosure. Thus, it is intended that the present disclosure cover such modifications and variations provided they come within the scope of the appended claims and their equivalents.

What is claimed is:

1. A method for treating thrombosis in a patient in need thereof, comprising:
administering a pharmaceutical composition comprising a proteolytic enzyme or mixture of proteolytic enzymes to the patient, and
administering a first balloon catheter to the patient.

2. The method according to claim 1, wherein the first balloon catheter comprises a balloon, a first tube, and a second tube, the first and second tubes each having an inlet located at the same side of the balloon,
wherein the first tube has an outlet inside of the balloon to inflate the balloon, and the second tube has an outlet located on another end of the balloon distant from the inlet to be located between the balloon and the thrombus.

3. The method according to claim 1, further comprising administering a second balloon catheter to the patient.

4. The method according to claim 1, wherein the pharmaceutical composition further comprises Lisini racemici acetylsalicylase.

5. The method according to claim 4, wherein the mixture of proteolytic enzymes comprises Krill enzymes.

6. A method of treating thrombosis in a patient, comprising:
a) blocking a vessel containing a thrombus downstream of said thrombus with a first balloon catheter to form a small volume between the first balloon catheter and the thrombus,
b) rinsing said volume,
c) administering a Krill enzyme solution into said volume until the thrombus is dissolved,
d) optionally, applying a stent into said vessel, and
e) optionally, applying a pharmaceutical composition comprising a proteolytic enzyme or mixture of proteolytic enzymes, Lysini racemici acetysalicylase, and a pharmaceutically acceptable excipient to said patient.

7. The method according to claim 6 wherein, in step a) the vessel is blocked upstream and downstream the thrombus to form two small volumes between the first balloon catheter and the thrombus, and a second balloon catheter and the thrombus;
wherein, in step c) a Krill enzyme solution is applied to said two small volumes until the thrombus is dissolved, and
wherein the two small volumes between the two balloon catheters are rinsed again after the thrombus is dissolved.

8. The method according to claim 6, wherein saline or Ringer solution is a rinsing agent for the rinsing of the volume between the first balloon catheter and the thrombus.

9. The method according to claim 5, wherein the Krill enzymes comprise three serine proteinases with trypsin-like activity and one serine proteinase with chymotrypsin-like activity.

10. The method according to claim 5, wherein the Krill enzymes comprise four exopeptidases, and wherein the four exopeptidases include two carboxypeptidases A and two carboxypeptidases B.

11. The method according to claim 9, wherein the three serine proteinases with trypsin-like activity include two endo/exopeptidases and one endopeptidase.

12. The method according to claim 5, wherein the krill enzymes reduce plaque on an arterial wall.

13. A pharmaceutical composition, comprising a proteolytic enzyme or mixture of proteolytic enzymes, Lysini racemici acetysalicylase, and a pharmaceutically acceptable excipient.

14. The pharmaceutical composition according to claim 13, wherein the mixture of proteolytic enzymes comprises Krill enzymes.

15. The pharmaceutical composition according to claim 13, comprising about 900 mg Lisini racemici acetylsalicylase.

16. The pharmaceutical composition according to claim 14, comprising about 60 units of Krill enzymes and about 900 mg Lisini racemici acetylsalicylase.

17. The pharmaceutical composition according to claim 14, wherein the Krill enzymes comprise three serine proteinases with trypsin-like activity and one serine proteinase with chymotrypsin-like activity.

18. The pharmaceutical composition according to claim 14, wherein the Krill enzymes comprise four exopeptidases.

19. The pharmaceutical composition according to claim 17, wherein the three serine proteinases with trypsin-like activity include two endo/exopeptidases and one endopeptidase.

20. The pharmaceutical composition according to claim 18, wherein the four exopeptidases include two carboxypeptidases A and two carboxypeptidases B.
